(19) United States
(12) Patent Application Publication
(10) Pub. No.: US 2010/0191063 A1
(43) Pub. Date: Jul. 29, 2010

(54) Portable otoscope device

(76) Inventor: Chun Chieh Hsu, Hsichih City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/320,541

(22) Filed: Jan. 29, 2009

(51) Int. Cl. A61B 1/267

(52) U.S. Cl. 600/200

(57) ABSTRACT

The present invention is a portable otoscope device that is mainly a single machine-operated structural design for convenient usage by users. It includes a grip styled main body with a protruding conical shape body set on the front end of its upper portion and a base portion set in the rear side, and it combines with a display panel. There is a control circuitry system set inside the main body that is including at least picture-taking and displaying mechanism. The protruding conical shape body is used to extend it into the patient's ears such that the images can be transferred to the display panel through the picture-taking mechanism. It then can provide a fast and simple surveillance and diagnosis, and it thus can obtain the practical advancement.

### PRIOR ART OF THE INVENTION

The known otoscope mainly is a design with optical typed structure that is provided to the otologists to survey the conditions inside the patient's ears. It is only for pure observation. By the combination of several lenses, the projected images are enlarged for visually diagnosing directly. Due to that it can only be provided for temporarily viewing while being used, cannot access the images and the viewing ranges are limited, it is not fit in with practical usage. Therefore, it is known that there is a design of picture-taking device in the ear to improve the flaws of the traditional optical typed otoscope, which utilizes a fixed typed computer main frame and screen, and combines with a picture-taking lens through a connecting wire to access the images by extending the lens with wire into the patent's ears then the images are displayed by computer's screen for viewing and getting the images. Although this function can obtain a certain degree of advancement, it still cannot be moved randomly and can only be used in the fixed locations besides the required gigantic equipment and higher cost. It thus is quite restricted while being used and waits to be improved.

### SUMMARY OF THE INVENTION

The object of the present invention is to provide a simple otoscope device with a portable structural design, which is convenient to the medical personnel and even can easily be single machine-used by the normal home users. And, it can access images rapidly then directly show them on the display panel such that the surveillance and diagnosis become more convenient, and the images can be stored for future recording and analyzing.

### BRIEF DESCRIPTION OF THE DRAWINGS

FIG. 1 is an outside appearance diagram of the present invention.

FIG. 2 is an outside appearance diagram of the present invention in another view angle.

FIG. 3 is a side view diagram of the present invention.

### DETAILED DESCRIPTION OF THE PREFERRED EMBODIMENT

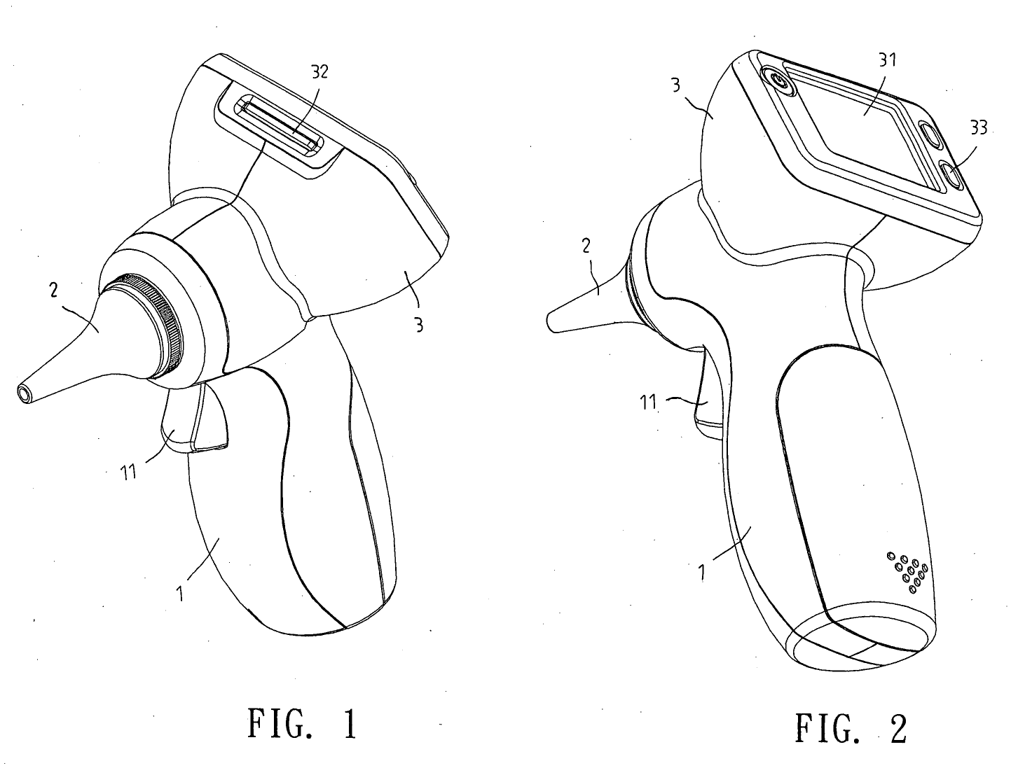

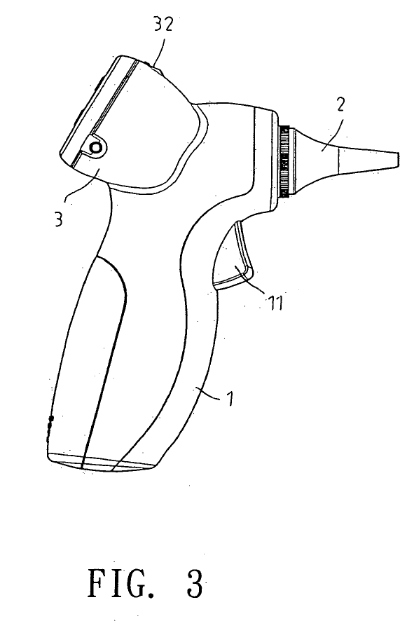

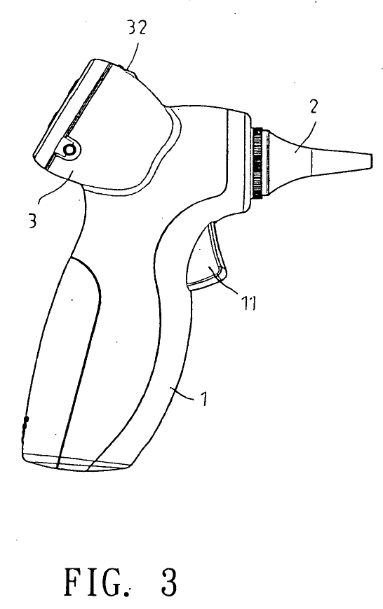

Please refer to those shown in FIGS. 1 to 3. The present invention includes a main body 1 whose modeling is a grip styled main body for easy holding with a protruding conical shape body 2 set in the front end of its upper portion and a base portion 3 set in the rear end, and is combining with a display panel 31. There is further a push-buttons portion 11 set inside the main body 1. And, a control circuitry system is set in the interior of the main body 1 that is able to be linked with at least a picture-taking and displaying mechanism and is able to be further connected with a control circuitry (the circuitry systems are well known and will not be shown as figures and described in detail) such as an ear or forehead temperature measuring system etc. There are further memory card slot 32 and several push buttons 33 set on the base portion 3.

While being used, the protruding conical shape body 2 is extended into the patient's ears. The accessed images by the picture-taking and displaying mechanism inside the main body 1 are enlarged then transferred to the display panel 31. It thus can provide the observer a conveniently direct observation and diagnosis on the panel 31. If there is a need for recording and storage after considering, the push-buttons portion 11 can be pressed to picture-take the images and store them in the memory card on the inserting slot 32, and view the historical stored images through the controlling of buttons. The images can also be outputted again on other equipments for further checking and diagnosing.

Due to that the present invention is an independent machine body with dedicate modeling, it thus can be provided to the normal home users or medical personnel for conveniently carrying out without the restriction of the requirement to connect with the computer or to be used at fixed locations. While checking the patients, the real situation inside the patient's ears can be observed more conveniently and rapidly by utilizing the picture-taking and showing directly on the panel to speed up the judgment of diagnosis and reduce the occurrence of the erroneous diagnosis.

Thus, the present invention can be provided to the users such that they can be used at will in any places and under any circumstances to fasten the checking and diagnosing for patients and can store the required images in time for further judgment, analysis or recording in the future to obtain the effects that currently existing known products can not achieve. And, in practical usage, the present invention can also utilize the internal ear or forehead temperature measuring system to measure the patient's body temperature. Therefore, it is also equipped with appended functions.

I claim:

1. A portable otoscope device including a grip styled main body with a protruding conical shape body set on its upper portion and a base portion set in the rear side, and combining with a display panel; a control circuitry system being set inside the main body that is including at least a picture-taking and displaying mechanism, and the protruding conical shape body is used to extend it into the patient's ears then picture-taking to get the images and show them directly on the display panel.

2. A portable otoscope device as claimed in claim 1, wherein a push-button portion is set in the main body and a memory card slot is set in the base portion to provide a combination with the memory card, the push-buttons can be operated to take the images and store them in the memory card; wherein several push-buttons are set at the side of panel to be operated to repeatedly view the historical images stored in the memory card.

3. A portable otoscope device as claimed in claim 1, wherein the control circuitry system can be connected with an ear or forehead temperature measuring mechanism.
